(19) United States
(12) Patent Application Publication
(10) Pub. No.: US 2020/0306095 A1
(43) Pub. Date: Oct. 1, 2020

(54) BODY COMPRESSION DEVICE WITH HOOK-AND-LOOP CLOSURE

(71) Applicant: Thonic Innovation, Monistrol sur Loire (FR)

(72) Inventor: Pierre GONON, Monistrol sur Loire (FR)

(21) Appl. No.: 16/759,835

(22) PCT Filed: Nov. 27, 2018
(86) PCT No.: PCT/IB18/59345

§ 371 Date: Apr. 28, 2020

(30) Foreign Application Priority Data
Nov. 30, 2017 (FR) 17/71297

(51) Int. Cl. A61F 13/08; A61F 5/058

(52) U.S. Cl. CPC A61F 13/085; A61F 13/10; A61F 5/05841

(57) ABSTRACT

Body compression device intended to be at least partially wound around a part of the body in order to exert a compression force on said part of the body, the device including a hook-and-loop fastening system with a plurality of loops arranged on a loop zone and a plurality of hooks arranged on a hooking surface, the hooking surface being elastic and capable of adopting at least two states: a first, unstretched state, in which the hooks are in immediate proximity to one another, and a second state, in which the hooking surface is substantially stretched, allowing it to be hooked onto the loops of the loop zone.

### CROSS-REFERENCE TO RELATED APPLICATIONS

This is a National Stage Entry into the United States Patent and Trademark Office from International Patent Application No. PCT/IB2018/059345, having an international filing date of Nov. 27, 2018, which claims priority to French Patent Application No. 17/71297, filed on Nov. 30, 2017, the entire contents of both of which are incorporated herein by reference.

### TECHNICAL FIELD OF THE INVENTION

The present invention relates to a body compression device intended to be at least partially wound over a part of the body and/or a limb in order to exert a compression force on said part of the body and/or said limb, said device comprising a fixing system with loops and hooks comprising a plurality of loops arranged on a loop zone and a plurality of hooks arranged on a hooking surface.

### STATE OF THE PRIOR ART

Elastic body devices, for example bands and/or orthoses, are primarily used to treat veino-lymphatic conditions leading notably to edemas, lymphoedemas or the like, and require treatment by splinting/compression. In order to best comply with the medical prescriptions, the level of tightening should be sufficient to allow a compression of the zones to be treated. Now, in practice, it is found that the patients apply the devices randomly, with highly variable levels of tightness, often rendering the treatments largely or totally ineffective.

When the treatment is performed by compression stockings or socks, the tightening effect obtained results from the size of the sock and the elasticity of the knit, but does not depend on the way in which the user puts on the sock. By contrast, medical bandages have to be fitted by the wearer or a carer, who winds the band by applying a certain tension. It is commonplace for the tension applied to be insufficient and/or irregular, particularly for bands of great length.

Moreover, a patient fitting a band on one of his or her limbs can experience certain difficulties in correctly applying it. Furthermore, when fitting a band on an arm, it is often difficult to, at the same time, perform the tightening, the winding and the fastening of the band.

Many examples illustrate the repeated efforts to achieve a device which is reliable, practical and inexpensive. Firstly, stretchable fabrics are known that have at least one “loop and hook” zone.

For example, the document FR1263391 describes a splinting band made of elastic fabric, intended to be wound around the human body. The band is produced in such a way that the degree of splinting can be varied by fixing its free end at determined locations and by using means that do not risk damaging the band. It is provided with at least one transverse strip made of special fabric, notably the fabric known in the trade as “Velcro”, in which the bristles have their ends bent back in the form of hooks, so as to be able to be selectively hooked in strips of fluffy tissue fixed onto the other face of said band. This hooking device seems to require the user to observe predefined zones for the hooking, which limits his or her hooking and therefore tightening freedom.

The document FR2806904 describes an elastic band intended to bandage a limb which comprises, at its free end in the direction of winding, a gripping transverse tongue of loop-and-hook fabric type situated on the face of the band turned toward the limb. This band is provided, on its face turned outward from the limb, with at least two receiving gripping transverse tongues, of loop-and-hook fabric type complementing the end tongue, positioned such that the proximal receiving tongue closest to the end of the band is situated at a distance therefrom that is less than or equal to the thinnest perimeter of the limb to be bandaged and the distal receiving tongue furthest away from the end of the band is situated at a distance therefrom greater than or equal to the perimeter of the thickest limb to be bandaged when the band is subjected to usage tension. The band can suit a large number of patients, but its tongue-based attachment system seems to present the same abovementioned drawbacks. Moreover, there is no means with which to ensure a suitable level of tightening of the band around the limb, which can result in an ineffective bandage because of inadequate tightening.

The document EP2250981 describes a multipurpose medical item comprising a strap of elastic fabric of elongate form characterized by a distal end and a proximal end, a distal surface and a proximal surface, in which at least a part of the proximal surface is, to the outside, provided with a textured surface of a first type, an absorbent patch fixed to the proximal surface of the strap forming a pocket between a hand piece attached to the proximal end of the strap, at least a part of the distal surface toward the outside provided with a textured surface of another kind, and at least a part of the proximal surface toward the outside provided with a textured surface of a third type at the proximal end thereof. The textured surface of the first kind and the textured surface of the second kind self-adhere by physical additions with one another forming a self-adhering hardening, and the textured surface of the third type is characterized by its capacity to self-adhere to the proximal surface of the strap and/or to the outer surface of the absorbent sheet adjoining the latter. This system does not seem to make it possible to have an accurate level of tightening.

The “loop-and-hook” type fixings are widely used in the medical field for fixing bands. Some fixings use molded hooks, despite their complexity to manufacture and their high cost.

For example, the document WO0168019 describe extendable fixings produced from a fixing tape in the form of a sheet which is cut into strips disposed longitudinally and separated transversely from one another. The strips spaced apart are fixed onto an elastic cloth in canvas form and constitute an extendable fixing. In some cases, the extendable fixing is formed continuously at the same time as the sheet-type fixing band. The molded hooks are well separated from one another so as to be always operational. However, the design of this fabric seems complex and costly. Furthermore, this system does not seem to make it possible to have an accurate level of tightening.

Some hooks of the “loop-and-hook” attachments are provided with stretchable materials, generally at precise locations.

For example, the document US2002022108 describes a fixing system comprising molded hooks separated from one another by free zones. The molding is done by rigid molds. To facilitate the formation of a product that is uniform, elastically extendable or flexible, the bands of fixing elements extend in the direction of the manufacturing machine. This hooking system seems effective, but, it appears complex and costly to manufacture. Furthermore, this system does not seem to make it possible to have an accurate level of tightening.

In the world of layers, some “loop-and-hook” hooking systems have extendable loops in order to be able to adjust the sizes for an effective hold.

For example, the document US2011152819 describes a layer fixing system comprising at least one elastomer connection tab disposed on a lateral part of the layer. The connection tab has a linked part comprising an elastomer expansion zone. The elastomer expansion zone comprises a non-elastic pitting substrate made of fibrous material with a plurality of tenfold plies and a zigzag array of elastomer threads disposed underlying the plies. The zigzag array is adapted to apply contraction forces to the stitching substrate in the lengthwise dimension of the connection tab. This hooking system is effective but does not seem to make it possible to have an accurate level of tightening. Furthermore, its design seems complex.

The document WO2017027830 describes an undergarment which comprises at least one panel of stretchable loop material. The panel of stretchable loop material is linked to a main body of the undergarment such that it can stretch around a side of a waist or hip of a wearer. The stretchable loop material is configured in such a way that it can be connected removably to a plurality of holding elements. The holding elements can be hooks of a loop-and-hook closure system. The undergarment is configured in such a way that it can be easily concealed under a garment and can be easily positioned and removed by a wearer. One method for arranging an undergarment consists in rolling or folding the main body of the undergarment and reattaching panels of stretchable loop material around the wound or folded part to cover the wound or folded part between the panels of stretchable loop material. This system appears comfortable and effective, but it does not seem to make it possible to have an accurate level of tightening.

Some hooking systems aim to obtain a maximum of stretchability and use an elastic fabric.

For example, the document WO2005004778 describes a ring-and-hook closure comprising an element with hooks and an element with rings. The element with hooks comprises a support with hooks and a plurality of hooks extending over said support with hooks. This support with hooks contains an extendable material. The element with rings comprises a support with rings and a plurality of rings extending over said support. This support with rings also contains an extendable material. This hooking system appears effective and comfortable, with a good rate of elasticity, but its design seems to be complex and costly. Furthermore, this system does not seem to make it possible to have an accurate level of tightening.

Finally, the document US2005/0132543 describes a fabric comprising raised zones interposed between zones provided with loops or hooks. The zones concerned can be unfolded but are not elastic.

All these different examples of devices show that there is a need for a solution that makes it possible to simplify the fitting of compression bandages, while ensuring that the tightening applied is sufficient to obtain the expected benefits.

To mitigate these various drawbacks, the invention provides various technical means.

### SUMMARY OF THE INVENTION

First of all, a first objective of the invention is to provide a powerful and ergonomic compression device.

Another objective of the invention is to provide a compression device that is simple to manufacture and inexpensive.

Yet another objective of the invention is to provide a compression device that makes it possible to improve the control of the level of tightening.

Yet another objective of the invention is to provide a compression device that makes it possible to increase the comfort of the user.

Finally, another objective of the invention is to provide a compression device that is easy to fit by a professional or by a novice.

For this, the invention provides a body compression device intended to be at least partially wound over a part of the body and/or a limb in order to exert a compression force on said part of the body and/or said limb, said device comprising a fixing system with loops and hooks comprising a plurality of loops arranged on a loop zone and a plurality of hooks arranged on a hooking surface, said hooking surface is elastic and can adopt at least two distinct states:
a first state, in which said hooking surface is unstretched and the very fine weave of the elastic fabric used forms a field of hooks with very high density and in particular that are very close to one another, such that the hooks cross one another and are nested in one another, preventing hooking with the loops of the loop zone, and
a second state, in which said hooking surface is stretched and in which the hooks are spaced apart and thus no longer nested in one another, rendering the hooks functional and allowing hooking with the loops of the loop zone.

According to such an architecture, the device, of so-called cohesive type, can be wound for example on a limb or a part of the body of a patient according to the part of the body to be treated. In the unstretched state of the hooking surface, the cooperation between the loops and the hooks is little or ineffective, whereas in the substantially stretched state of the hooking surface, the cooperation between the loops and the hooks is effective, rendering the hooking functional and effective. Indeed, in the unstretched state, the hooks are nested in one another and are totally or almost nonoperational. Conversely, in the stretched state, the hooking surface is elongate, which makes it possible for the hooks to be no longer nested in one another. This disposition of the hooks is made possible by the enlarging of the area of the hooking surface. The hooks therefore move away from one another and the hooking system becomes operational. The architecture makes it possible to ensure that the tightening force is sufficient to allow the therapeutic action. Furthermore, the tightening force is well distributed over all the length of the band or of the orthosis.

According to an advantageous embodiment, the device comprises at least one portion made of fabric comprising a first face and a second face, characterized in that at least one loop zone is arranged on said first face of the fabric, and at least one hooking surface is arranged on said second face of the fabric.

To facilitate the implementation of the device, the disposition of the loops on one of the faces and of the hooks on the other face allows for simple winding and hooking. The design and industrialization are also simplified.

According to another advantageous embodiment, the hooking surface is mono-elastic, the direction of elasticity corresponding substantially to the axis of winding of the device on said part of the body.

As a variant, the hooking surface is bi-elastic.

If the band is not wound with sufficient force, the hooking surface is in the unstretched state, making fixing impossible. This makes it possible to avoid having a compression device fitted without tension which would be partially or wholly ineffective. Conversely, an adequate tightening allows the hooking zone to be in the stretched position, thus freeing the hooks and allowing the device to be fixed. A conformal level of tightening ensures that the band or the orthosis is wound with a sufficient level of tightening to allow the compression of the limb to be treated.

Advantageously, the hooking surface and the loop zone are produced in a same type of fabric.

This implementation is particularly simple and economic and nonaggressive for the skin of the patient and the other textiles.

Also advantageously, the body device comprises an active compression part provided to exert a compression force distributed over the limb or the part of the body to be treated.

According to an advantageous embodiment, said active part and the fixing system are produced with the same fabric.

A one-piece body device is thus constructed that is simple, esthetic and particularly effective.

According to another advantageous embodiment, the fixing system is made of a piece with the active part.

The continuity thus obtained provides a comfortable and ergonomic product.

Advantageously, the active part is composed of a material or fabric that is different from the fabric of which the fixing system is composed.

According to an advantageous embodiment, the device is a splinting/compression band.

According to another advantageous embodiment, the device is a splinting/compression orthosis.

Advantageously, the potential stretch rate of the hooking surface (20) surface lies between 25% and 100%, and more preferentially between 30% and 75% and even more preferentially between 30% and 50%.

According to yet another advantageous embodiment, the hooking surface is composed of a loop zone transformed into a hooking surface, for example by a laser attack on the plurality of loops.

This type of implementation makes it possible to produce a product of simple design, the industrialization of which is greatly simplified, particularly when it is implemented with one and the same fabric.

### DESCRIPTION OF THE FIGURES

All the production details are given in the following description, complemented by FIGS. 1a to 8b, presented purely as nonlimiting examples, and in which:

FIGS. 1a to 1d are schematic representations illustrating an example of fabric comprising loop zones (FIG. 1a), of which one loop zone makes it possible to generate a hooking surface (FIG. 1b) shown in unstretched mode (FIG. 1c) and in stretched mode (FIG. 1d);

FIGS. 2a and 2b schematically illustrate the two faces of an example of compression device;

FIGS. 3, 4a to 4e and 5 are schematic representations showing the variant productions of a compression device of splinting band type; and

FIGS. 6a to 8b are schematic representations showing various examples of production of the compression device of splinting orthosis type.

### DETAILED DESCRIPTION OF THE INVENTION

Example of Implementation of a Fixing System with Loops and Hooks

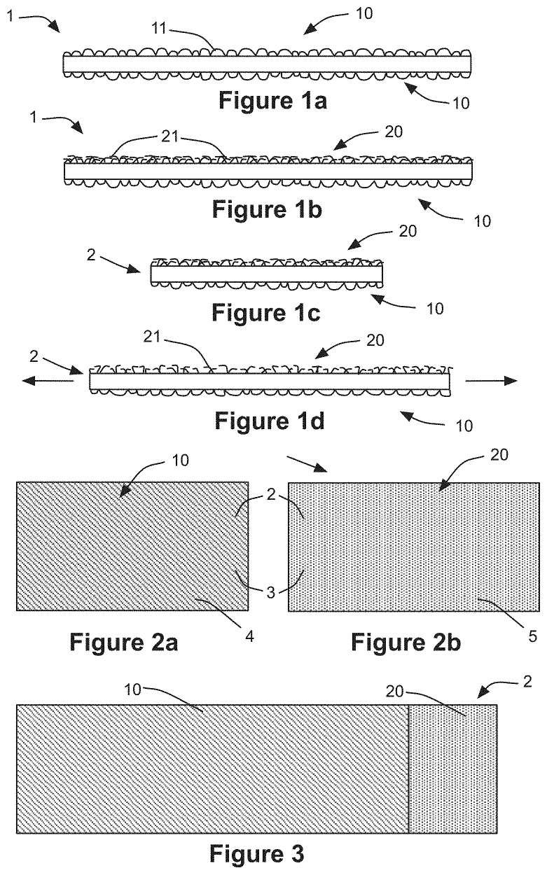

FIGS. 1a to 1d schematically illustrate, from an example of an elastic fabric 1 initially comprising loop zones 10 on each of its faces, an example of implementation of a fixing system 3 with loops and hooks used in compression devices described in relation to FIGS. 2a to 8b.

In its initial state, the fabric 1 comprises, on each side, a plurality of loops 11 formed in loop zones 10 as presented in FIG. 1a.

FIG. 1b schematically illustrates the transformation performed in the loop zone 10 of the top face to obtain a hooking zone 20. The threads forming the loops 11 are treated, for example using a laser attack, to transform them at least partially into hooks 21. The treatment implemented on the fabric makes it possible for at least a portion of the loops of the elastic fabric to be modified. For example, the loops are cut into at least two parts, the remaining parts of the loop remaining fixed to the weft of the fabric, and form hooks 21. The treatment makes it possible to obtain inclined or bent back hook ends.

The very fine weave of the elastic fabric used makes it possible to obtain a “field” of hooks with very high density and in particular that are very close to one another, such that the hooks cross one another. In such a configuration, corresponding to the unstretched mode, the hooks are partially or totally ineffective for catching any loops. FIG. 1c illustrates an example of arrangement of the hooks 21 distributed on a hooking surface 20 in the unstretched state.

The fabric 1 is stretchable at least in the longitudinal direction. This feature makes it possible to space the hooks 21 apart and therefore for them to be no longer nested in one another when the hooking surface 20 is in its stretched state, as presented in FIG. 1d. In this state, the hooks are functional or effective, and can grip or catch any loops. The level of stretching of the hooking surface to favor a good operation of the hooks is advantageously greater than 30% and more preferentially lies between 30% and 50%.

The fixing system 3 is thus noteworthy in that it comprises two distinct states, intrinsically linked to the elasticity of the fabric of which the system is composed.

This system moreover provides a particularly high level of fixing because, once the loops are hooked into the hooks (in stretched mode), the fabric is then relaxed to revert to a substantially unstretched state. In this state, the hooks are once again close to one another and thus create a kind of locking of the hooks, making the separation of the loops and hooks more difficult, and achievable through a “relatively high” separation effort (?). To detach the loops and hooks, the hooking surface is preferably stretched. This precaution makes it possible to facilitate the separation of the loops and hooks and protects the loops and the hooks against premature damage.

Moreover, the high density of the hooks and their random arrangement makes it possible to optimize to the maximum the hooking of the fixing system 3.

### First Example of Product Using a Fixing System with Loops and Hooks: Splinting/Compression Band

FIGS. 2a to 5 schematically illustrate examples of splinting bands 2 comprising at least portions produced with a fabric 1 similar to that used in FIGS. 1a to 1d to produce the fixing system 3 previously described.

The body device 2 comprises at least one portion made of fabric 1 comprising two faces 4 and 5. A loop zone 10 is arranged on the first face 4, schematically represented in FIG. 2a. A hooking surface 20 is arranged on the second face 5, schematically represented in FIG. 2b.

In this way, the winding of the splinting band is particularly easy because no matter where the free end of the hooking surface 20 arrives on the loop zone 10, hooking is possible. Indeed, with this embodiment, any part of the second face 5 can be fixed to any part of the first face 4, provided that the hooking surface 20 is stretched, to allow the disposition of the hooks 21 which can be fixed to the loops 11 of the loop zone 10.

Moreover, this feature makes it possible to have a continuous fixing system 3 over all the length of the splinting band, for example, when the user partly overlaps the band in the winding.

In order to effectively treat symptoms such as bad venous and/or lymphatic circulation for example, the splinting band must be applied with a level of tightening that is sufficient, and if possible applied uniformly over all the length of the band. The fixing system 3, by virtue of its two states, makes it possible to avoid inadequate tightening because the hooking surface 20 must be stretched for the hooks 21 to be able to grip the loops 11 and therefore hold the device in place.

FIG. 3 schematically shows another example of splinting band 2. In this example, the fixing system 3 is configured with a hooking surface 20 arranged at the free end of the band. The rest of the band is composed of loops 10, as illustrated in FIG. 3. This configuration allows the user to fix the band on any loop zone 10. The device is therefore suited to all morphologies and practically all the parts of the body to be treated.

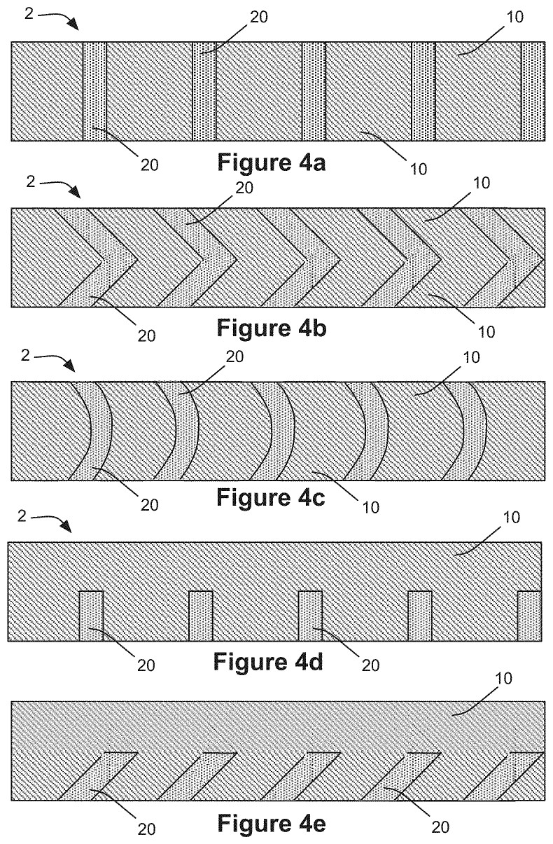

In the exemplary embodiment of FIG. 4a, loop zones 10 and hooking surfaces 20 are arranged alternately along the band. This configuration makes it possible to multiply the hooking sectors in order to have a better hold without having a surface made only of hooks 21.

FIGS. 4b and 4c present variant embodiments in which loop zones 10 are arranged in chevron or circular arc form.

FIGS. 4d and 4e present variant embodiments in which loop zones 10 are arranged over a fraction of the width of the band. In the examples illustrated, the fraction corresponds to half the width. As a variant, different fractions are provided, for example covering between ⅕ and ⅘ of the width of the band. These embodiments are advantageous in the case where the band is wound around a limb of the patient, being implemented with a partial overlap from one turn to another.

In order to reduce the costs and simplify the implementation, the loop zone 10 and the hooking surface 20 can be produced in the same material and/or the same type of fabric.

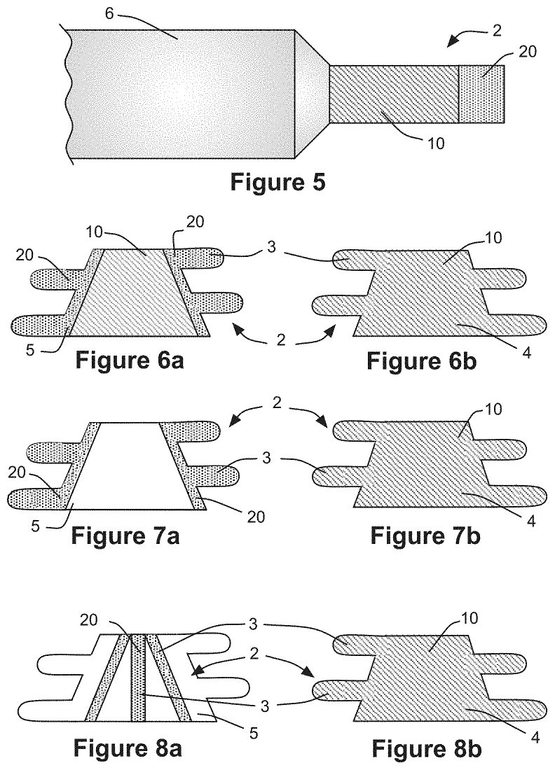

As a variant, as illustrated in FIG. 5, the splinting band 2 comprises an active part 6 provided to exert a compression effort once the band is suitably installed. The active part can, as a variant, be produced in the same material as the fixing system 3. This feature makes it possible to have a single-piece production, in order to simplify the manufacture and reduce the cost while having an effective device. The fixing system 3 of the splinting band can also be made of a piece with the active compression part 6, which makes it possible to obtain a product that is simple to design, comfortable and effective.

Furthermore, this feature makes it possible to have a single part, without connection between the different loop zones 10 and the hooking surfaces 20, which, in an extreme use for example, could embrittle the fixing system 3.

The splinting force of the bands is measured using a dynamometer. For example, after three 0% to 30% fatigue cycles, the force necessary to elongate the elastic band to 30% is recorded on the last cycle (according to the ITFH method). Based on their splinting force, the bands can be split up into different levels, such as, for example, 20 to 45 cN/cm, 46 to 100 cN/cm, 101 to 160 cN/cm, and greater than 160 cN/cm.

### Second Example of Product Using a Fixing System with Loops and Hooks: Splinting/Compression Orthosis

FIGS. 6a to 8b schematically illustrate exemplary embodiments in which the body compression device 2 is a splinting orthosis. The orthosis comprises a cylindrical central body and lateral tabs, provided to facilitate the fixing to a limb of the body by winding around the latter.

In all the examples, the figure on the right illustrates a first face 4 and the figure on the left presents the second face 5. In all these examples, the first face 4 is covered with a loop zone 10.

In the first example (FIG. 6a), the second face 5 comprises a loop zone 10 formed in the central body and hooking surfaces 20, on each side and on the lateral tabs.

In the second example (FIG. 7a), the second face 5 comprises a free zone formed in the central body and hooking surfaces 20, on each side and on the lateral tabs.

Finally, the last example (FIG. 8a) provides a plurality of hooking zones 20 distributed over the central body and free surfaces, on each side and on the lateral tabs. Multiple variant embodiments of the orthosis are possible, for example with lateral tabs on only one side or without lateral tab.

The operation and the implementation of the examples of orthosis illustrated are similar to the splinting bands previously described, except for the fact that the orthoses form a winding over a single turn. As previously described for the splinting bands, the two-state fixing system 3 makes it possible to ensure that the orthoses are applied with a sufficient level of tightening. In case of insufficient tightening or in the absence of tightening, the fixing system remains inoperative and the orthosis cannot be fixed.

1. A body compression device intended to be at least partially wound over a part of the body and/or a limb in order to exert a compression force on said part of the body and/or said limb, said device comprising a fixing system with loops and hooks comprising a plurality of loops arranged on a loop zone and a plurality of hooks arranged on a hooking surface, wherein said hooking surface is elastic and can adopt at least two distinct states:
a first state, in which said hooking surface is unstretched and the very fine weave of the elastic fabric used forms a field of hooks with very high density and in particular that are very close to one another, such that the hooks cross one another and are nested in one another, preventing hooking with the loops of the loop zone, and
a second state, in which said hooking surface is stretched and in which the hooks are spaced apart and thus no longer nested in one another, rendering the hooks functional and allowing hooking with the loops of the loop zone.

2. The body compression device of claim 1, wherein the device comprises at least one portion made of fabric comprising a first face and a second face, wherein at least one loop zone is arranged on said first face of the fabric, and at least one hooking surface is arranged on said second face of the fabric.

3. The body compression device of claim 1, wherein said hooking surface is mono-elastic, the direction of elasticity corresponding substantially to the axis of winding of the device on said part of the body.

4. The body compression device of claim 1, wherein the hooking surface and the loop zone are produced in a same type of fabric.

5. The body compression device of claim 1, comprising an active compression part provided to exert a compression force distributed over the limb or the part of the body to be treated.

6. The body compression device of claim 5, wherein said active compression part and the fixing system are produced with the same fabric.

7. The body compression device of claim 5, wherein the fixing system is made of a piece with the active part.

8. The body compression device of claim 5, wherein the active part is composed of a material or fabric different from the fabric of which the fixing system is composed.

9. The body compression device of claim 1, wherein the device is a splinting/compression band.

10. The body compression device of claim 1, wherein the device is a splinting/compression orthosis.

11. The body compression device of claim 1, wherein the potential stretch rate of the hooking surface lies between 25% and 100%.

12. The body compression device of claim 1, wherein the hooking surface is composed of a loop zone transformed into a hooking surface.
